(19) United States
(12) Patent Application Publication
(10) Pub. No.: US 2020/0144668 A1
(43) Pub. Date: May 7, 2020

(54) LITHIUM ION SECONDARY BATTERY, MANUFACTURING METHOD OF LITHIUM ION SECONDARY BATTERY, AND ELECTROLYTE FOR LITHIUM ION SECONDARY BATTERY

(71) Applicant: ENVISION AESC ENERGY DEVICES LTD., Sagamihara-shi, Kanagawa (JP)

(72) Inventors: Hideaki SASAKI, Sagamihara-shi (JP); Satoru HIRAKAWA, Sagamihara-shi (JP)

(73) Assignee: ENVISION AESC ENERGY DEVICES LTD., Sagamihara-shi, Kanagawa (JP)

(21) Appl. No.: 16/608,529

(22) PCT Filed: Apr. 9, 2018
(86) PCT No.: PCT/JP2018/014911

§ 371 Date: Oct. 25, 2019

(30) Foreign Application Priority Data
Apr. 26, 2017 (JP) 2017-087254

(51) Int. Cl. H01M 10/0567; H01M 10/0525; H01M 4/525; H01M 10/44; C07D 327/00

(52) U.S. Cl. CPC H01M 10/0567; H01M 10/0525; H01M 2004/028; H01M 10/446; C07D 327/00; H01M 4/525

(57) ABSTRACT

In a lithium ion secondary battery including a positive electrode including a positive electrode active material containing a lithium nickel complex oxide, a cyclic sulfonic acid ester (al) which contains at least two sulfonyl groups in a molecule and a compound (a2) which contains only one sulfonyl group in a molecule and of which an energy level of a highest occupied molecular orbital calculated by a PM3 method is −11.2 eV or less are used in an electrolyte. In addition, by charging such a battery, a film including a sulfur atom is formed on at least a portion of a surface of the positive electrode active material.

### TECHNICAL FIELD

The present invention relates to a lithium ion secondary battery, a manufacturing method of a lithium ion secondary battery, and an electrolyte for a lithium ion secondary battery.

### BACKGROUND ART

Since a lithium ion secondary battery can realize high energy density, the lithium ion secondary battery is widely applied to not only mobile communication devices or personal computers but also various uses such as large electricity storage power sources, automobile power sources, and various precision instruments or power sources for motive machinery. In order to further improve performance, various examinations and proposals are continuously made about materials/compositions such as positive electrodes, negative electrodes, and electrolytes. For example, use of a lithium nickel complex oxide as a positive electrode active material is actively considered (refer to Patent Documents 1 to 4).

### RELATED DOCUMENT

### Patent Document

[Patent Document 1] Japanese Patent Application No. 5063948

[Patent Document 2] Japanese Unexamined Patent Publication No. 2010-64944

[Patent Document 3] Japanese Unexamined Patent Publication No. 2014-222624

[Patent Document 4] Japanese Unexamined Patent Publication No. 2015-90857

### SUMMARY OF THE INVENTION

### Technical Problem

A lithium ion secondary battery including a positive electrode active material containing a lithium nickel complex oxide can theoretically realize high electric potential and high capacity.

However, according to the examination of the present inventors, it was found that, in a case where a charging and discharging cycle of the battery is repeated under a condition of a relatively high temperature of around 45° C. (in the assumption of outdoor use), for example, there is room for improvement, from a viewpoint of reduction of discharge capacity, an increase in a battery volume due to generation of gases, an increase in charge transfer resistance, and the like.

The present invention is made in consideration of the circumstances. That is, even in a case where the lithium ion secondary battery including a positive electrode active material containing a lithium nickel complex oxide was repeatedly used (charge and discharge) under a condition of a relatively high temperature (around 45° C.), objects of the present invention is to decrease reduction of a charge capacity, to suppress an increase in a battery volume due to generation of gases, and to suppress an increase in charge transfer resistance.

### Solution to Problem

The present inventors carried out intensive examination to solve the above problem. As a result, it was found that, in a case where an additive is contained in an electrolyte of a battery, an energy level of a highest occupied molecular orbital of the additive was related to the battery performance. And the problem was solved by selecting an appropriate additive having the energy level of the highest occupied molecular orbital of the additive as a design guideline.

Specifically, the problem was solved by the following first to fourth inventions.

A first invention is a lithium ion secondary battery including a positive electrode including a positive active material containing a lithium nickel complex oxide, a negative electrode, and an electrolyte, in which the electrolyte includes a cyclic sulfonic acid ester (a1) which contains at least two sulfonyl groups in a molecule and a compound (a2) which contains only one sulfonyl group in a molecule and of which an energy level of a highest occupied molecular orbital calculated by a PM3 method is −11.2 eV or less.

A second invention is a lithium ion secondary battery including a positive electrode including a positive electrode active material containing a lithium nickel complex oxide, a negative electrode, and an electrolyte, in which a film containing a sulfur atom is present on at least a portion of a surface of the positive electrode active material, and the electrolyte includes a compound (a2) which contains only one sulfonyl group in a molecule and of which an energy level of a highest occupied molecular orbital calculated by a PM3 method is −11.2 eV or less.

A third invention is a manufacturing method of a lithium ion secondary battery of the “second invention”, the invention including a step of assembling a non-charged lithium ion secondary battery including (i) the electrolyte including the compound (a1) which contains two or more sulfonyl groups in a molecule and the compound (a2) which contains only one sulfonyl group in a molecule and of which an energy level of a highest occupied molecular orbital calculated by a PM3 method is −11.2 eV or less, (ii) the positive electrode including a positive electrode active material containing a lithium nickel complex oxide, and (iii) the negative electrode; and a step of performing charge on the non-charged lithium ion secondary battery, reacting the positive electrode active material with the compound (a1) included in the electrolyte, and forming a film containing a sulfur atom on at least a portion of the surface of the positive electrode active material.

A fourth invention is an electrolyte for lithium ion secondary battery including a positive electrode including a positive electrode active material containing a lithium nickel complex oxide, the electrolyte including a compound (a1) which contains two or more sulfonyl groups in a molecule and a compound (a2) which contains only one sulfonyl group in a molecule and of which an energy level of a highest occupied molecular orbital calculated by a PM3 method is −11.2 eV or less.

Here, the first to fourth inventions and embodiments thereof have close relevance to each other.

To put it simply, when the lithium ion secondary battery of the first invention is charged, a film containing a sulfur atom is formed on at least a portion of a surface of the positive electrode. That is, it is possible to “manufacture” the lithium ion secondary battery of the second invention from the lithium ion battery of the first invention. The third invention regards the “manufacture” as an invention of a manufacturing method. In addition, the fourth invention is an invention that is particularly focused on the electrolyte among the composition of the lithium ion secondary battery of the first invention.

The lithium ion secondary battery of the second invention is preferably manufactured by a manufacturing method of the third invention, but may be manufactured by other manufacturing methods. In addition, the lithium ion secondary battery of the second invention is not necessarily manufactured from the lithium ion secondary battery of the first invention.

The relevance of the first to fourth inventions is also appropriately mentioned in description of the embodiments of the present invention.

### Advantageous Effects of Invention

According to the present invention, even in a case where the lithium ion secondary battery including a positive electrode active material containing a lithium nickel complex oxide was repeatedly used (charge and discharge) under a condition of a relatively high temperature (around 45° C.), it is possible to suppress reduction of a charge capacity, an increase in a battery volume due to generation of gases, and an increase in charge transfer resistance.

### DESCRIPTION OF EMBODIMENTS

Hereinafter, embodiments of the present invention will be described in detail.

In the present specification, “a to b” of a numerical value range indicate a or more and b or less, unless particularly refused.

In the following, first, the embodiments of the lithium ion secondary battery of the first invention will be described in detail. Hereinafter, the embodiments of the second to fourth inventions will be described, but in the embodiments of the second to fourth inventions, description of the matters in common with the embodiment of the first invention will be appropriately omitted.

In addition, in the following, the embodiment of the first invention is referred to as “first embodiment”, the embodiment of the second invention is referred to as “second embodiment”, the embodiment of the third invention is referred to as “third embodiment”, and the embodiment of the fourth invention is referred to as “fourth embodiment”.

### First Embodiment

A lithium ion secondary battery according to a first embodiment is a lithium ion secondary battery including a positive electrode including a positive electrode active material containing a lithium nickel complex oxide, a negative electrode, and an electrolyte, in which the electrolyte includes a cyclic sulfonic acid ester (a1) which contains at least two sulfonyl groups in a molecule and a compound (a2) which contains only one sulfonyl group in a molecule and of which an energy level of a highest occupied molecular orbital calculated by a PM3 method is −11.2 eV or less.

In the present specification, the cyclic sulfonic acid ester (a1) which contains at least two sulfonyl groups in a molecule is simply referred to as “compound (a1)”. In addition, the compound (a2) which contains only one sulfonyl group in a molecule and of which the energy level of the highest occupied molecular orbital calculated by the PM3 method is −11.2 eV or less is simply referred to as “compound (a2)”.

A mechanism in which a discharge amount is hardly reduced even when charging and discharging the lithium ion secondary battery under a relatively high temperature (around 45° C.), an increase in a battery volume due to generation of gases can be suppressed, and an increase in charge transfer resistance can also be suppressed is not necessarily clear. However, according to the hypothesis, knowledge, and the like of the present inventors, explanation is possible as follows.

One of the causes of performance deterioration or generation of gases due to charge and discharge of the lithium ion secondary battery is generally considered to be that components in the electrolyte are decomposed on the electrode (negative electrode or positive electrode). In particular, since a nickel atom has high reactivity with an organic compound in the electrolyte compared to other metal atoms, in a case where a positive electrode active material including a lithium nickel complex oxide as a positive electrode active material was used, it is considered that performance deterioration or generation of gases can be easily problematic. In addition, since chemical reaction generally proceeds under a high temperature environment, it is considered that decomposition of components in the electrolyte further easily proceeds.

It is generally known that, regarding the compound (a1), a film is formed on a surface of a negative electrode at the time of charge. However, according to the knowledge of the present inventors, in a case where a lithium nickel complex oxide was used in a positive electrode, due to specific reactivity with the nickel and the compound (a1), the compound (a1) also reacts with a positive electrode, a “film” including a sulfur atom is formed on a surface of the positive electrode. This is supported by the following experiment/analysis result by the present inventors.

(Experiment/Analysis Result)

Through a charge and discharge cycle evaluation of the lithium ion secondary battery using an electrolyte including a compound corresponding to the compound (a1), using a lithium nickel complex oxide as a positive electrode active material, the following (i) to (iv) results are obtained.

(i) In a relatively initial stage of the charge and discharge cycle, there came to a state in which the compound (a1) almost did not remain in the electrolyte. That is, the compound (a1) was “consumed” in an initial stage of the charge and discharge cycle.

(ii) When the battery after the charge and discharge cycle was decomposed, and the surface of the positive electrode was analyzed by TOF-SIMS (time-of-flight secondary ion mass spectrometry), a sulfur oxide component (SOx) was detected.

(iii) When the battery after the charge and discharge cycle was decomposed, and the surface of the positive electrode was subjected to wide scan measurement of XPS (X-ray photoelectron spectroscopy), a peak of S(2s) was detected in the vicinity of 230 eV and a peak of S(2p) was detected in the vicinity of 170 eV.

(iv) From the XPS measurement (detailed scan), a peak (164 eV) derived from an S atom in a reduced state was detected on a surface of the positive electrode. (Since the S in a reduced state is present, it is considered that Ni—S bond is present.)

When a film including a sulfur atom is formed on the surface of the positive electrode, it is considered that the electrolyte does not directly come into contact with nickel and the like in the positive electrode active material. In this regard, decomposition of the electrolyte is suppressed, and as a result, it is considered that reduction of discharge capacity, an increase in charge transfer resistance, generation of gases, and the like are suppressed.

On the other hand, the compound (a2) has relatively low energy level of the highest occupied molecular orbital (HOMO). This means that electron donicity of the compound (a2) is small.

In this regard, it is considered that the compound (a2) having small electron donicity does not react as actively as the compound (a1) on the surface of the positive electrode, reacts little by little with nickel and the like of the positive electrode in repeating charge and discharge cycles, and gradually forms a film on the positive electrode. That is, even after the charge and discharge cycles were repeated in multiple times and the compound (a1) was consumed, the film on the surface of the positive electrode is maintained in a constant state, and deterioration of the film is suppressed by reacting the compound (a2) little by little.

As described above, the film is formed on the surface of the positive electrode, and the film is maintained even when charge and discharge are repeated, due to complementary action between the compound (a1) and the compound (a2). As a result, under a condition in which decomposition reaction of the electrolyte is easily caused, that is, when a lithium ion secondary battery including a positive electrode active material containing a lithium nickel complex oxide is repeatedly charged and discharged at a relatively high temperature (around 45° C.), reduction of discharge capacity, an increase in a battery volume due to generation of gases, an increase in charge transfer resistance, and the like are suppressed.

Each constituent of the lithium ion secondary battery according to the first embodiment will be described.

### [Electrolyte]

The lithium ion secondary battery according to the first embodiment includes an electrolyte, and the electrolyte contains a compound (a1) and a compound (a2). In addition, the electrolyte preferably contains a lithium salt, a solvent, and the like.

In the present specification, not only those in a liquid form having fluidity but also those having substantially no fluidity such as “polymer gel electrolyte” which is known in the present technical field are included in the concept of the “electrolyte”. In the present embodiment, the electrolyte is preferably in a liquid form having fluidity.

Compound (a1)

The compound (a1) is not particularly limited as long as the compound is a cyclic sulfonic acid ester having at least two sulfonyl groups in a molecule, and examples thereof include a compound represented by the following general formula (1).

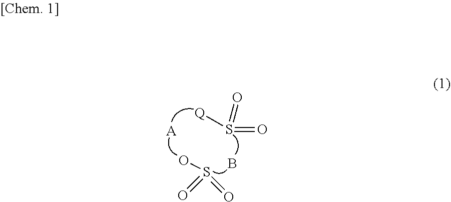

In the general formula (1), Q represents an oxygen atom, a methylene group, or a single bond.

A represents an alkylene group, a carbonyl group, a sulfinyl group, a fluoroalkylene group, and a divalent group to which an alkylene unit or a fluoroalkylene unit bind through an ether bond.

B represents an alkylene group, a fluoroalkylene group, or an oxygen atom.

The alkylene group of A preferably has 1 to 5 carbon atoms, and may be non-substituted or may further contain a substituent.

The fluoroalkylene group of A preferably has 1 to 6 carbon atoms, and may be non-substituted or may further contain a substituent.

The divalent group to which an alkylene unit or a fluoroalkylene unit bind through an ether bond, of A preferably has 2 to 6 carbon atoms.

The alkylene group and the fluoroalkylene group of B may be non-substituted or may further contain a substituent.

The alkylene group and the fluoroalkylene group of B preferably have 1 to 6 carbon atoms, more preferably have 1 to 3 carbon atoms, and further more preferably have 1 carbon atom.

In the general formula (1), in a case where Q is a single bond, a carbon molecule and S constituting A is configured to form a C—S single bond.

In addition, in the general formula (1), the preferable carbon atoms of A and B indicate the number of carbons constituting a ring, and do not include the number of carbon atoms contained in a side chain.

Specific examples of the compound (a1) are shown in the following table, but the compound (a1) is not limited to the specific examples shown in the following table.

TABLE 1

| Compound | Chemical |
|---|---|
| No. | structure |
| 1 | 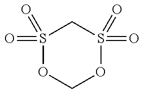 |
| 2 | 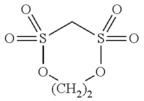 |
| 3 | 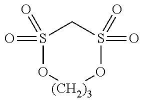 |
| 4 | 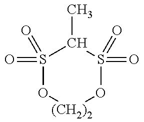 |
| 5 | 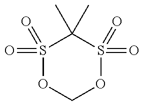 |
| 6 | 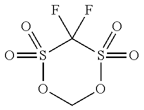 [SMILES: O=S1(=O)OCOS(=O)(=O)C1(F)F] |
| 7 | 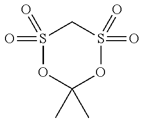 |
| 8 | 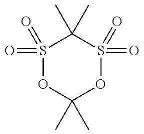 |
| 9 | 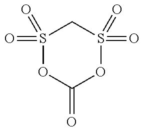 [SMILES: O=C1OS(=O)(=O)CS(=O)(=O)O1] |
| 10 | 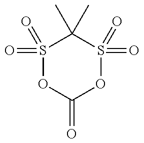 |
| 11 | 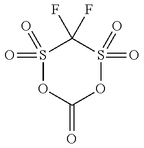 [SMILES: O=C1OS(=O)(=O)C(F)(F)S(=O)(=O)O1] |
| 12 | 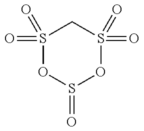 [SMILES: O=S1OS(=O)(=O)CS(=O)(=O)O1] |
| 13 | 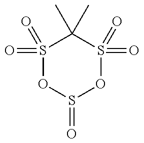 |
| 14 | 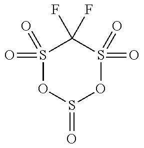 [SMILES: O=S1OS(=O)(=O)C(F)(F)S(=O)(=O)O1] |
| 15 | 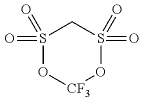 |
| 16 | 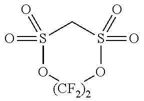 |
| 17 | 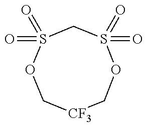 |
| 18 | 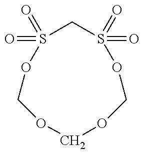 [SMILES: O=S1(=O)CS(=O)(=O)OCOCOCO1] |
| 19 | 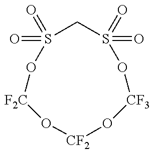 |
| 20 | 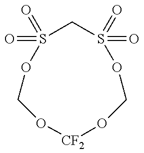 [SMILES: O=S1(=O)CS(=O)(=O)OCOC(F)(F)OCO1] |
| 21 | 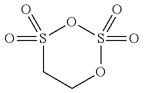 |
| 22 | 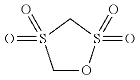 |

A content of the compound (a1) in the electrolyte, in the lithium ion secondary battery according to the first embodiment is 0.1 to 5.0% by mass, preferably 0.5 to 5.0% by mass, more preferably 1.0 to 3.0% by mass, and further more preferably 1.0 to 2.0% by mass, for example, based on the total content of the electrolyte. By setting the content of the compound (a1) in the electrolyte within the numerical value range, it is possible to obtain a film with an appropriate thickness on the electrode.

Compound (a2)

The compound (a2) is not particularly limited as long as the compound is a compound containing only one sulfonyl group in a molecule and of which an energy level of the highest occupied molecular orbital (HOMO) calculated by the PM3 method is −11.2 eV or less. The energy level of HOMO is preferably −11.8 eV or more.

Here, a calculation result (numerical value) by the PM3 method is a result obtained by designating keywords “PM3 EF PRECISE GNORM=0.05 NOINTER GRAPHF MMOK” by the software MOPAC6.03 in the present specification.

A preferable aspect of the compound (a2) is a compound of which the energy level of the lowest unoccupied molecular orbital (LUMO) calculated by the PM3 method is 0 to 0.2 eV. By using the compound (a2) having LUMO in the numerical value range, consumption of the compound (a2) in the negative electrode is suppressed. Accordingly, even when the charge and discharge cycles are repeated, the compound (a2) remains in multiple numbers, which is desirable.

The compound (a2) is preferably a compound that has a ring structure, and contains only one —SO₂-structure in the ring structure. More preferably, a compound represented by the following general formula (2) is exemplified.

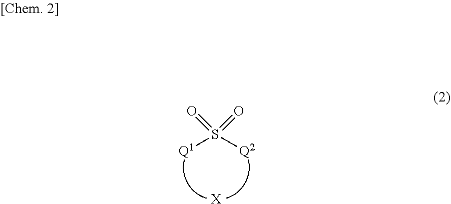

In the general formula (2), Q¹ and Q² each independently represents a single bond or an oxygen atom.

X represents an alkylene chain or a fluoroalkylene chain. This may contain an ether bond in a chain.

The alkylene chain or the fluoroalkylene chain of X preferably have 3 to 7 carbon atoms. In addition, examples thereof include an alkyl group (methyl group, ethyl group, and the like), a hydroxy group, a halogen atom, and the like. Examples of the substituent include an alkyl group (methyl group, ethyl group, and the like), a hydroxy group, a halogen atom, and the like.

In the following general formula (2), in a case where Q¹ is a single bond, a carbon molecule and S constituting X is configured to form a C—S single bond. Similarly, in a case where Q² is a single bond, a carbon molecule and S constituting X is configured to form a C—S single bond.

The compound represented by the general formula (2) preferably has 5 to 8 membered rings constituted by S-Q¹-X-Q², and more preferably has 5 to 6 membered rings (that is, preferably a compound having 5 to 6 membered rings). The “membered rings” indicates the number of carbon and hetero atoms that directly constitute a ring structure, and the atoms of the side chain of the ring structure is not counted.

Regarding types of the compound having only one sulfonyl group in a molecule, the energy level of HOMO and LUMO is shown in Table 1. Among these, those of which the energy level of HOMO is −11.2 eV or less (PS, 24BS, TMS) correspond to the compound (a2).

The compound (a2) is not limited to the specific compounds (PS, 24BS, TMS).

TABLE 2

| Compound | Abbreviation | HOMO/eV | LUMO/eV | Note |
|---|---|---|---|---|
| 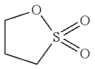 | PS | −11.5055 | 0.06069 | Corresponds to compound (a2). |
| 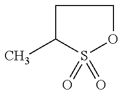 | 24BS | −11.47562 | 0.10748 | Corresponds to compound (a2). |
| 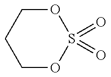 | TMS | −11.46933 | 0.0045 | Corresponds to compound (a2). |
| 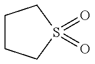 | SL | −10.94996 | 0.12193 | Does not corresponds to compound (a2). |
| 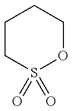 | 14BS | −11.09494 | 0.00507 | Does not corresponds to compound (a2). |

A concentration of the compound (a2) is preferably 1.0% by mass to 6.0% by mass, more preferably 1.0% by mass to 4.0% by mass, and further more preferably 1.0% by mass to 2.0% by mass, based on the total content of the electrolyte. By setting the concentration of the compound (a2) within the amount, even after the charge and discharge cycles were repeated, it is considered that a necessary and sufficient amount of the compound (a2) remains in the electrolyte, and the film of the positive electrode is easily maintained in an appropriate state.

Lithium Salt

The electrolyte preferably includes a lithium salt.

The lithium salt is not particularly limited. Any known lithium salt can be used, and the lithium salt may be selected depending on the type of the positive electrode or the type of the negative electrode. In addition, two or more kinds may be used in combination.

Examples thereof include LiClO₄, LiBF₄, LiPF₆, LiCF₃SO₃, LiCF₃CO₂, LiAsF₆, LiSbF₆, LiB₁₀Cl₁₀, LiAlCl₄, LiCl, LiBr, LiB (C₂H₅)₄, CF₃SO₃Li, CH₃SO₃Li, LiCF₃SO₃, LiC₄F₉SO₃, LiN(SO₂F)₂, a lithium lower fatty acid carboxylate, and the like. Among these, LiBF₄, LiPF₆, and LiN(SO₂F)₂ are preferable from a viewpoint of availability.

A concentration of the lithium salt in the electrolyte (the total amount in a case where the electrolyte includes a plurality of types of lithium salts) is generally 0.1 to 3.0 mol/L, and preferably 0.5 to 2.0 mol/L based on the total content of the electrolyte. By setting the concentration of the lithium salt within the numerical value range, it is possible to obtain sufficient conductivity.

Solvent

The electrolyte typically includes a solvent.

The solvent preferably contains a non-aqueous solvent. The solvent is not particularly limited, and examples thereof include carbonates such as ethylene carbonate (EC), propylene carbonate (PC), butylene carbonate (BC), dimethyl carbonate (DMC), diethyl carbonate (DEC), methylethyl carbonate (MEC), and vinylene carbonate (VC); lactones such as γ-butyrolactone and γ-valerolactone; ethers such as trimethoxy methane, 1,2-dimethoxy ethane, diethyl ether, 2-ethoxy ethane, tetrahydrofuran, and 2-methyl tetrahydrofuran; sulfoxides such as dimethyl sulfoxide; oxolanes such as 1,3-dioxolane, and 4-methyl-1,3-dioxolane; nitrogenous substances such as acetonitrile, nitromethane, formaldehyde, and dimethyl formaldehyde; organic acid esters such as methyl formate, methyl acetate, ethyl acetate, butyl acetate, methyl propionate, and ethyl propionate; phosphate triester and diglymes; triglymes; sulfolanes such as sulfolane and methyl sulfolane; oxazolidinones such as 3-methyl-2 oxazolidinone; and sultones such as 1,3-propane sultone, 1,4-butane sultone, and naphtha sultone. One kind thereof may be independently used, or two or more kinds thereof may be used in combination.

The electrolyte preferably does not include moisture. That is, the electrolyte preferably does not include moisture other than moisture inevitably contained through manufacture or use.

### [Positive Electrode]

The lithium ion secondary battery according to the first embodiment includes a positive electrode including a positive electrode active material containing a lithium nickel complex oxide.

The positive electrode typically has a structure including a current collector layer, and a layer including the positive electrode active material (positive electrode active material layer) on a single surface or both surfaces of the current collector layer. In addition, the positive electrode active material layer preferably includes a positive electrode active material, a binder resin, and a conductive auxiliary agent.

Positive Electrode Active Material Including Lithium Nickel Complex Oxide

The positive electrode active material is not particularly limited as long as it is a positive electrode active material including a lithium nickel complex oxide. Examples thereof include a complex oxide such as lithium nickel complex oxide, lithium nickel manganese complex oxide, lithium nickel cobalt complex oxide, lithium nickel aluminum complex oxide, lithium nickel cobalt aluminum complex oxide, lithium nickel manganese cobalt complex oxide, lithium nickel manganese aluminum complex oxide, and lithium nickel cobalt manganese aluminum complex oxide.

Among these, a complex oxide including at least one element selected from the group consisting of cobalt and aluminum is preferable. That is, a lithium nickel cobalt complex oxide, a lithium nickel aluminum complex oxide, and a lithium nickel cobalt aluminum complex oxide are preferable.

The lithium nickel complex oxide is a complex oxide of which composition formula is represented by LiₓNi_{1-y}M_{y}O₂ (M includes at least one metal selected from the group consisting of Co, Fe, Ti, Cr, Mg, Al, Cu, Ga, Mn, Zn, Sn, B, V, Ca, and Sr, and satisfies 0.05≤x≤1.2, 0·y·0.5). Among these, M preferably includes Co and/or Al. In addition, y is preferably 0.1≤y≤0.4, and more preferably 0.1≤y≤0.2.

A plurality of the positive electrode active materials may be used in combination. In this case, a plurality of the lithium nickel complex oxides may be used, or a lithium nickel complex oxide and a material which is not the lithium nickel complex oxide may be used in combination. In a case of the latter, from a viewpoint of easily obtaining an effect (high electric potential and the like) due to the lithium nickel complex oxide, the content of the lithium nickel complex oxide in the total content of the positive electrode active material is preferably 50% by mass or more, and more preferably 80% by mass or more.

An average particle diameter of the positive electrode active material is preferably 1 μm or more, more preferably 2 μm or more, further more preferably 5 μm, and from a viewpoint of input and output properties or electrode preparation (smoothness of electrode surface and the like), preferably 80 μm or less, more preferably m or less, and further more preferably 20 μm or less. Here, the average particle diameter means a particle diameter (median diameter: D50) at 50% of an integrated value in particle distribution (in terms of volume) by a laser diffraction scattering method. By setting the average particle diameter within the numerical range, side reaction at the time of charge and discharge is suppressed and deterioration of charge and discharge efficiency is suppressed.

The content of the positive electrode active material is preferably 85 parts by mass to 99.4 parts by mass, more preferably 90.5 parts by mass to 98.5 parts by mass, and further more preferably 90.5 parts by mass to 97.5 parts by mass, when the total content of the positive electrode active material is set as 100 parts by mass. From this, sufficient storage and release of lithium can be expected.

Binder Resin

Binder resin is appropriately selected, and not particularly limited. For example, in a case where N-methyl-pyrrolidone (NMP) is used as a solvent, it is possible to use those generally used such as polytetrafluoroethylene (PTFE), polyvinylidene fluoride (PVDF), and the like.

A content of the binder resin is preferably 0.1 part by mass to 10.0 parts by mass, more preferably 0.5 parts by mass to 5.0 parts by mass, and further more preferably 1.0 part by mass to 5.0 parts by mass, when the total content of the positive electrode active material layer is set as 100 parts by mass. When the content of the binder resin is within the range, balance between coating properties of an electrode slurry, binding properties of a binder, and battery characteristics are further excellent. In addition, when the content of the binder resin is the upper limit value or less, a proportion of the electrode active material becomes great, and a capacity per electrode mass preferably becomes great. When the content of the binder resin is the lower limit value or more, electrode exfoliation is suppressed, which is preferable.

Conductive Auxiliary Agent

The conductive auxiliary agent is not particularly limited as long as the conductive auxiliary agent improves conductivity of the electrode. Examples thereof include carbon black, ketchen black, acetylene black, natural graphite, artificial graphite, carbon fiber, and the like. One kind of the conductive auxiliary agents thereof may be independently used, or two kinds thereof may be used in combination.

The content of the conductive auxiliary agent is preferably 0.5 parts by mass to 5.0 parts by mass, more preferably 1.0 part by mass to 4.5 parts by mass, and further more preferably 1.5 parts by mass to 4.5 parts by mass, when the total content of the positive electrode active material layer is set as 100 parts by mass. When the content of the conductive auxiliary agent is within the range, balance between coating properties of electrode slurry, binding properties of a binder, and battery characteristics are further excellent. In addition, when the content of the conductive auxiliary agent is the upper limit value or less, a proportion of the electrode active material becomes larger, and capacity per electrode mass preferably becomes bigger. When the content of the conductive auxiliary agent is the lower limit value or more, conductivity of the electrode becomes more favorable, which is preferable.

In addition, preferable aspects related to the positive electrode will be described.

Density of Positive Electrode Active Material Layer

Density of the positive electrode active material layer is not particularly limited, but is preferably 2.0 to 3.6 g/cm³, for example. When the density is within the numerical value range, discharge capacity is improved at a time of use at a high discharge rate, which is preferable.

Thickness of Electrode Active Material Layer

A thickness of the electrode active material layer is not particularly limited, and can be appropriately set depending on a desired characteristic. For example, from a viewpoint of energy density, it is possible to set the thickness to be thick, and from a viewpoint of output characteristics, it is possible to set the thickness to be thin. The thickness of the positive electrode active material layer can be appropriately set within a range of 10 to 250 μm, preferably 20 to 200 μm, and more preferably 40 to 180 μm, for example.

Current Collector Layer

In a current collector layer, it is possible to use aluminum, stainless steel, nickel, titanium, or an alloy thereof, and from a viewpoint of price, availability, electrochemical stability, and the like, aluminum is particularly preferable. In addition, a shape of the current collector layer is also not particularly limited, but it is preferable to use a foil shape, a flat plate shape, or a mesh shape with a thickness of 0.001 to 0.5 mm.

Preparation Method of Positive Electrode

A preparation method of a positive electrode is not particularly limited.

Typically, (i) first, electrode slurry obtained by dispersing or dissolving a positive electrode active material, a binder resin, and a conductive auxiliary agent in an appropriate solvent is prepared, (ii) subsequently, the electrode slurry is coated on a single surface or both surfaces of the current collector layer and dried to provide a positive electrode active material layer, (iii) and then it is possible to obtain a positive electrode by pressing the electrode active material layer formed on the single surface or both surfaces of the current collector layer with the current collector layer.

### [Negative Electrode]

The lithium ion secondary battery according to the first embodiment includes a negative electrode. The negative electrode typically has a structure including a current collector layer, and a negative electrode active material layer on a single surface or both surfaces of the current collector layer. The negative active material layer generally includes a negative electrode active material, and a binding agent or a conductive auxiliary agent depending on the necessity.

As the negative electrode active material, graphite, amorphous carbon, silicon, silicon oxide, metal lithium, and the like are preferably exemplified, but the negative electrode active material is not particularly limited as long as it is a material capable of storing or discharging lithium.

An average particle diameter of the negative electrode active material is preferably 1 μm or more, more preferably 2 μm or more, further more preferably 5 μm or more, and from a viewpoint of input and output characteristics or electrode preparation (smoothness of electrode surface and the like), is preferably 80 μm or less, and more preferably 40 μm or less. Here, the average particle diameter means a particle diameter (median diameter: D50) at 50% of an integrated value in particle distribution (in terms of volume) by a laser diffraction scattering method. By setting the average particle diameter within the numerical value range, side reaction at the time of charge and discharge is suppressed and deterioration of charge and discharge efficiency is suppressed.

The negative electrode active material layer may contain a conductive auxiliary agent or a binding agent depending on the necessity. As the conductive auxiliary agent or the binding agent, it is possible to use the same agent as those capable of being used on the above-described positive electrode active material layer. In addition, as the current collector layer, it is possible to use stainless steel, nickel, titanium, or the alloy thereof.

### [Separator]

The lithium ion secondary battery according to the first embodiment preferably includes a separator. The separator is mainly made of a porous membrane made of resin, woven fabric, non-woven fabric, and the like, and as the resin components, it is possible to use a polyolefin resin such as polypropylene and polyethylene, a polyester resin, an acrylic resin, a styrene resin, or a nylon resin. In particular, a polyolefin-based microporous membrane is excellent in ion permeability, and performance of physically isolating the positive electrode and the negative electrode, and thus is preferable. In addition, the separator may form a layer including inorganic particle, and as the inorganic particle, an insulating oxide, nitride, sulfide, carbide, and the like can be exemplified. Among these, a separator including TiO₂ or Al₂O₃ is preferable.

### [Exterior Container]

The lithium ion secondary battery according to the first embodiment is preferably put in an appropriate exterior container. As the exterior container, it is possible to use a case made of a flexible film or a can case. From a viewpoint of weight reduction, it is preferable to use a flexible film. As the flexible film, those in which a resin layer is provided on a front and back surface of a metal layer serving as a base can be used. As the metal layer, it is possible to select those having barrier properties such as preventing leaking of an electrolyte and penetration of moisture from outside, and aluminum, stainless steel, and the like can be used. On at least one surface of the metal layer, a thermal fusion resin layer such as modified polyolefin is provided. The exterior container is formed by opposing the thermal fusion resin layers of the flexible film to each other, and performing thermal fusion of the periphery of a portion accommodating an electrode laminated body. It is possible to provide a resin layer such as a nylon film and a polyester film on an exterior surface made of a surface on which a thermal fusion resin layer is formed and a surface on an opposite side.

### Second Embodiment

The lithium ion secondary battery according to a second embodiment is a lithium ion secondary battery including a positive electrode including a positive electrode active material including a lithium nickel complex oxide, a negative electrode, and an electrolyte, in which a film including a sulfur atom is present on at least a portion of a surface of the positive electrode active material, and the electrolyte contains a compound (a2) which contains only one sulfonyl group in a molecule and of which an energy level of the highest occupied molecular orbital calculated by the PM3 method is −11.2 eV or less.

Each composition of the lithium ion secondary battery according to the second embodiment will be described.

### [Positive Electrode]

Regarding the positive electrode including a positive electrode active material containing a lithium nickel complex oxide in the lithium ion secondary battery according to the second embodiment, a film including a sulfur atom is present on at least a portion of the surface of the positive electrode active material.

Although description was also made in the ion secondary battery according to the first embodiment, it is considered that as such a film is present, the electrolyte does not directly come into contact with nickel and the like in the positive electrode active material. In this regard, decomposition of the electrolyte is suppressed, and as a result, it is considered that generation of gases is suppressed.

As a method of forming such a film, there is a method of preparing the above-described lithium ion secondary battery according to the first embodiment, and then charging thereof, for example. That is, by reacting the compound (a1) in the electrolyte of the lithium ion secondary battery according to the first embodiment with a positive electrode surface, at least a portion of the film of the surface of the positive electrode active material is formed. The method will be described in the description of a “third embodiment” to be described later.

A method of forming a film may be a method other than charging the lithium ion secondary battery according to the first embodiment. For example, it may be a method in which (i) first, by preparing a dedicated device, a positive electrode including a positive electrode active material containing a lithium nickel complex oxide is reacted with an appropriate sulfur compound, and a positive electrode on which a film is formed is obtained by an electrochemical method, and thereafter, (ii) the positive electrode on which a film is formed is taken, and is used as a positive electrode material of the lithium ion secondary battery according to the second embodiment.

The type of a positive electrode active material, the average particle diameter, the content of the positive electrode active material in the total content of the positive electrode active material layer, and the like, in the lithium ion secondary battery according to the second embodiment, are the same as those of the aspect in the lithium ion secondary battery according to the first embodiment, and a preferable aspect is the same.

In addition, the type of a binder resin and the content, the type of a conductive auxiliary agent and the content, the density of the positive electrode active material layer, the thickness of the electrode active material layer, and the type, the form, the thickness, and the like of a current collector layer, of the positive electrode in the lithium ion secondary battery according to the second embodiment are the same as those of the aspect in the lithium ion secondary battery according to the first embodiment, and a preferable aspect is the same.

### [Negative Electrode]

The lithium ion secondary battery according to the second embodiment includes a negative electrode. As the negative electrode, it is possible to use the same negative electrode as those described in the lithium ion secondary battery according to the first embodiment, and a preferable aspect is the same.

### [Electrolyte]

The lithium ion secondary battery according to the second embodiment includes an electrolyte, and the electrolyte contains a compound (a2) which contains only one sulfonyl group in a molecule and of which an energy level of the highest occupied molecular orbital calculated by the PM3 method is −11.2 eV or less.

Although description was also made in the lithium ion secondary battery according to the first embodiment, it is considered that the compound (a2) reacts with nickel and the like of the positive electrode little by little on the surface of the positive electrode and becomes a film on the positive electrode little by little, during repeating the charge and discharge cycle. As a result, it is considered that the film on the surface of the positive electrode is easily maintained in a constant state, and deterioration of the film is suppressed.

As the compound (a2), it is possible to use the same as those described in the lithium ion secondary battery according to the first embodiment, and a preferable aspect (compound structure, concentration, and the like) is also the same.

The electrolyte preferably contains a lithium salt, a solvent, and the like, in addition to the compound (a2). It is possible to use the same material as those described in the lithium ion secondary battery according to the first embodiment, and a preferable aspect is also the same. In addition, although this is the same as the lithium ion secondary battery according to the first embodiment, the electrolyte may be in a liquid form having fluidity, or may be a gel electrolyte having no fluidity.

The electrolyte may include the compound (a1) described in the lithium ion secondary battery according to the first embodiment.

### [Separator]

The lithium ion secondary battery according to the second embodiment preferably includes a separator. As the separator, it is possible to use the same as those described in the lithium ion secondary battery according to the first embodiment.

### [Exterior Container]

The lithium ion secondary battery according to the second embodiment is preferably put in an appropriate exterior container. As the exterior container, it is possible to use the same as those described in the lithium ion secondary battery according to the first embodiment.

### Third Embodiment

A third embodiment is a manufacturing method of the lithium ion secondary battery according to the “second embodiment”, the method including a step of assembling a non-charged lithium ion secondary battery including (i) an electrolyte including a compound (a1) which contains two or more sulfonyl groups in a molecule, and a compound (a2) which contains only one sulfonyl group in a molecule and of which an energy level of the highest occupied molecular orbital calculated by a PM3 method is −11.2 eV or more, (ii) a positive electrode including a positive electrode active material containing a lithium nickel complex oxide, and (iii) a negative electrode; and a step of performing charge on the non-charged lithium ion secondary battery, reacting the positive electrode active material with the compound (a1) included in the electrolyte, and forming a film containing a sulfur atom on at least a portion of the surface of the positive electrode active material.

Here, the “non-charged lithium ion secondary battery” is typically the lithium ion secondary battery according to the first embodiment described above. (This does not mean that the above-described lithium ion secondary battery according to the first embodiment indicates only a non-charged battery.)

That is, when assembling the above-described non-charged and discharged lithium ion secondary battery, those preferably capable of being used as the compound (a1) or the compound (a2), the composition of the electrolyte to be prepared, the positive electrode, the negative electrode, and other materials (including also the separator or the exterior container) used in assembling, the use amount of each material, and the like are the same as those described in the lithium ion secondary battery according to the first embodiment.

When charge and discharge are performed on the above-described non-charged and discharged lithium ion secondary battery, the compound (a1) in the electrolyte reacts with the positive electrode, and a film including a sulfur atom on the surface of the positive electrode is formed. That is, it is possible to manufacture the above-described lithium ion secondary battery according to the second embodiment.

The method of charge and discharge is not particularly limited as long as a film including a sulfur atom is formed on the surface of the positive electrode. For example, there is a method in which charge is performed on the above-described non-charged and discharged lithium ion secondary battery at a constant current of 0.05 to 1 C until it reaches 4.0 to 4.2 V, subsequently, charge is performed at a constant voltage of 4.0 to 4.2 V such that the total time combined with the above-described constant current charge is 1.5 to 20 hours, and then constant current discharge is performed under a condition of 0.05 to 1 C until it reaches 2.5 to 3.0 V. 1 C indicates a current value at which charge is completed in 1 hour, and can be theoretically obtained from materials of the positive electrode and the negative electrode, the use amount, and the like. According to the knowledge of the present inventors, the positive electrode active material containing a lithium nickel complex oxide and the compound (a1) specifically react with each other. Therefore, when charge and discharge is performed at least one time, a film containing a sulfur atom is formed on the surface of the positive electrode.

### Fourth Embodiment

A fourth embodiment is an electrolyte for a lithium ion secondary battery including a positive electrode including a positive electrode active material containing a lithium nickel complex oxide, the electrolyte containing a compound (a1) which contains two or more sulfonyl groups in a molecule and a compound (a2) which contains only one sulfonyl group in a molecule and of which an energy level of a highest occupied molecular orbital calculated by a PM3 method is 11.2 eV or less.

As described in the first embodiment, when such an electrolyte is used, it is possible to form a film including a sulfur atom on at least a portion of a surface of the positive electrode active material in the lithium ion secondary battery including the positive electrode including the positive electrode active material containing the lithium nickel complex oxide. In addition, even when charge and discharge cycles are repeated, the film is continuously maintained in an appropriate state, due to complementary action between the compound (a1) and the compound (a2). As a result, it is considered that reduction of discharge capacity, an increase in charge transfer resistance, generation of gases, and the like are suppressed.

Those capable of being preferably used as the compound (a1) and the compound (a2) in the electrolyte according to the fourth embodiment, components (lithium salt, solvent, and the like) other than the compound (a1) and the compound (a2) in the electrolyte, the use amount (content) of each component, the matter that the electrolyte preferably does not contain moisture, and the like are the same as those described as the “electrolyte”, in the lithium ion secondary battery according to the first embodiment.

Hereinabove, although embodiments of the present invention have been described, these are examples of the present invention, and various constituents other than the above-described ones can be employed. In addition, the present invention is not limited to the embodiments, and modifications, improvements, and the like within a range capable of achieving the object of the present invention are included in the present invention.

### EXAMPLES

The present invention will be described in detail using examples and comparative examples, but the present invention is not limited to the examples.

### Example 1

Preparation of Positive Electrode

94% by mass of LiNi_{0.8}Co_{0.15}Al_{0.05}O₂ as a positive electrode active material, 3% by mass of carbon as a conductive auxiliary agent, and 3% by mass of polyfluorinated vinylidene as a binder were mixed with one another, and the resultant material was added with an N-methyl pyrrolidone solvent and further mixed to prepare a positive electrode slurry. This was coated on both surfaces of an aluminum foil used as a current collector, dried, and roll-pressed to prepare a positive electrode. Adjustment was performed such that a coating amount of the positive electrode active material layer was 25 mg/cm², and the density was 3.4 g/cm³.

Preparation of Negative Electrode

97% by mass of graphite as a negative electrode active material, 2% by mass of styrene/butadiene rubber as a binder, and 1% by mass of carboxyl methyl cellulose were mixed by adding ion exchange water to prepare a negative electrode. This was coated on both surfaces of a copper foil used as a current collector, dried, and roll-pressed to prepare a negative electrode. Adjustment was performed such that a coating amount of the negative electrode active material layer was 16 mg/cm², and density was 1.5 g/cm³.

Preparation of Electrolyte

(1) 30 vol % of ethylene carbonate (EC), 20 vol % of diethylene carbonate (DEC), and 50 vol % of ethyl methyl carbonate (EMC) were mixed with one another to prepare a base electrolyte.

(2) Lithium hexafluorophosphate (LiPF₆) as a lithium salt was added to the base electrolyte obtained in (1) and mixed therein.

An addition amount was determined such that a concentration in the electrolyte injected into the lithium ion secondary battery was 1.0 mol/L.

(3) Methylene methane disulfonate as the compound (a1) (Compound No. 1 shown in the above Table 1, hereinafter, also abbreviated as “MMDS”) and 1,3 propane sultone (“PS” shown in the above Table 2) as the compound (a2) were added to the solution obtained in the above (2), and mixed therein. The addition amount was determined such that a mass concentration in the electrolyte injected into the lithium ion secondary battery was 0.5% by mass and 1.5% by mass, respectively.

Preparation of Lithium Ion Secondary Battery

The positive electrode and the negative electrode prepared above were stacked through a separator made of polypropylene to prepare a laminated body, and the laminated body was accommodated in the laminate exterior body. After that, the above-described prepared electrolyte was injected, and the laminate-type lithium ion secondary battery was prepared. Here, the prepared laminate-type lithium ion secondary battery is described as “cell”.

### Example 2

Except that the concentration of the compound (a2) in the electrolyte was 1.0% by mass, the laminate-type lithium ion secondary battery was prepared in the same manner as that of Example 1.

### Example 3

Except that the concentration of the compound (a2) in the electrolyte was 1.5% by mass, the laminate-type lithium ion secondary battery was prepared in the same manner as that of Example 1.

### Example 4

Except that the concentration of the compound (a2) in the electrolyte was 2.0% by mass, the laminate-type lithium ion secondary battery was prepared in the same manner as that of Example 1.

### Example 5

Except that 24BS shown in the above Table 2 was used, instead of 1,3 propane sultone of the compound (a2), in the electrolyte, the laminate-type lithium ion secondary battery was prepared in the same manner as that of Example 3.

### Example 6

Except that TMS shown in the above Table 2 was used, instead of 1,3 propane sultone of the compound (a2), in the electrolyte, the laminate-type lithium ion secondary battery was prepared in the same manner as that of Example 3.

### Comparative Example 1

Except that 14BS shown in the above Table 2 was used, instead of 1,3 propane sultone of the compound (a2), in the electrolyte, the laminate-type lithium ion secondary battery was prepared in the same manner as that of Example 3.

### Comparative Example 2

Except that SL shown in the above Table 2 was used, instead of 1,3 propane sultone of the compound (a2), in the electrolyte, the laminate-type lithium ion secondary battery was prepared in the same manner as that of Example 3.

### Comparative Example 3

Except that only methylene methane disulfonate (MMDS) was used as an additive in the electrolyte, and the concentration was 3% by mass in the electrolyte injected into the lithium ion secondary battery, the laminate-type lithium ion secondary battery was prepared in the same manner as that of Example 1.

### Comparative Example 4

Except that only PS shown in the above Table 2 was used as an additive in the electrolyte, and the concentration was 3% by mass in the electrolyte injected into the lithium ion secondary battery, the laminate-type lithium ion secondary battery was prepared in the same manner as that of Example 1.

### Comparative Example 5

Except that only SL shown in the above Table 2 was used as an additive in the electrolyte, and the concentration was 3% by mass in the electrolyte injected into the lithium ion secondary battery, the laminate-type lithium ion secondary battery was prepared in the same manner as that of Example 1.

### Comparative Example 6

Except that 14BS shown in the above Table 2 was used as an additive in the electrolyte, and the concentration was 3% by mass in the electrolyte injected into the lithium ion secondary battery, the laminate-type lithium ion secondary battery was prepared in the same manner as that of Example 1.

### Comparative Example 7

Except that only 24BS shown in the above Table 2 was used as an additive in the electrolyte, and the concentration was 3% by mass in the electrolyte injected into the lithium ion secondary battery, the laminate-type lithium ion secondary battery was prepared in the same manner as that of Example 1.

### Comparative Example 8

Except that only TMS shown in the above Table 2 was used as an additive in the electrolyte, and the concentration was 3% by mass in the electrolyte injected into the lithium ion secondary battery, the laminate-type lithium ion secondary battery was prepared in the same manner as that of Example 1.

### Comparative Example 9

Except that only vinylene carbonate (abbreviated as VC) was used as an additive in the electrolyte, and the concentration was 3% by mass in the electrolyte injected into the lithium ion secondary battery, the laminate-type lithium ion secondary battery was prepared in the same manner as that of Example 1. An energy level of HOMO of VC is −10.21311 (eV), and an energy level of LUMO is 0.08932 (eV).

### Comparative Example 10

Except that only fluoroethylene carbonate (FEC) was used as an additive in the electrolyte, and the concentration was 3% by mass in the electrolyte injected into the lithium ion secondary battery, the laminate-type lithium ion secondary battery was prepared in the same manner as that of Example 1. An energy level of HOMO of FEC is −10.37876 (eV), and an energy level of LUMO is −0.29602 (eV).

### Comparative Example 11

Except that only succinic anhydride (SA) was used as an additive in the electrolyte, and the concentration was 3% by mass in the electrolyte injected into the lithium ion secondary battery, the laminate-type lithium ion secondary battery was prepared in the same manner as that of Example 1. An energy level of HOMO of SA is −11.51757 (eV), and an energy level of LUMO is 0.16935 (eV). That is, those which did not contain a sulfonyl group but the energy levels of HOMO and LUMO satisfied the condition of the compound (a2) were selected.

Through the following procedure, evaluation of initial charge and discharge (formation of film on surface of positive electrode) and performance was evaluated on each lithium ion secondary battery prepared in the above-described Examples and Comparative Examples.

### [Initial Charge and Discharge]

The laminate-type lithium ion secondary battery prepared in the above-described Examples and Comparative Examples was charged at a constant current of 0.2 C until it reached 4.2 V, and after that, constant voltage charge at 4.2 V was performed for a total of 6.5 hours. Through the initial charge, a film containing a sulfur atom was formed on the surface of the positive electrode.

By decomposing the laminate-type lithium ion secondary battery of Example 1 after charge and discharge and performing XPS analysis of the positive electrode, it was confirmed that the film containing a sulfur atom was formed on the surface of the positive electrode.

### [Discharge Capacity Maintenance Rate]

Cycle characteristics were evaluated, using the laminate-type secondary battery on which the above-described initial charge was performed. Specifically, in an atmosphere of a temperature of 45° C., charge and discharge cycles of a charge rate of 1.0 C, a discharge rate of 1.0 C, a charge termination voltage of 4.20 V, and a discharge termination voltage of 2.5 V were repeated. By comparing discharge capacity after 300 cycles and discharge capacity of second cycle, a capacity maintenance rate was obtained.

The evaluation result was shown in Table 3. In the evaluation, a case where the capacity maintenance rate was more than 70% was indicated as GOOD, and a case where the capacity maintenance rate was 70% or less was indicated as POOR.

### [Increase in Volume (Amount of Generated Gas)]

By comparing cell volume after 300 cycles and cell volume after second cycle, a volume change rate, that is, an amount of generation of gases was obtained. The cell volume was performed using the Archimedes' method.

The evaluation result was shown in Table 3. In the evaluation, a case where volume change was less than 1% was indicated as GOOD, and volume change was 1% or more was indicated as POOR.

### [Resistance Increase Rate]

By comparing charge transfer resistance after 300 cycles and charge transfer resistance of second cycle, a resistance increase rate was obtained. The charge transfer resistance was obtained from a size of the arc by performing AC impedance measurement at room temperature (frequency: 10 kHz to 0.05 Hz, voltage amplitude: 10 mV) and drawing a Cole-Cole plot.

The evaluation result was shown in Table 3. In the evaluation, a case where volume change was less than 3% was indicated as GOOD, and volume change was 3% or more was indicated as POOR.

From Table 3, in a case where both of the additive corresponding to the compound (a1) and the additive corresponding to the compound (a2) were used, a favorable result was obtained in all of three performance evaluations in that the discharge capacity maintenance rate was more than 70%, the volume change was less than 1%, and the resistance increase rate was less than 3%.

That is, the lithium ion secondary batteries of Examples 1 to 4 in which MMDS was used as the compound (a1) and PS was used as the compound (a2) showed favorable characteristics in that the discharge capacity maintenance rate was more than 70%, the volume change was less than 1%, and the resistance increase rate was less than 3%. In addition, similarly in Examples 5 and 6 in which 24BS or TMS was used instead of PS, as the compound (a2), the favorable result was obtained.

Measurement data was elaborately analyzed, and as a result, it was found that, when the MMDS concentration was increased, the capacity maintenance rate tended to slightly decrease, but the capacity change (generation of gases) and the resistance increase rate were suppressed. From the balance of performance, it is considered that the concentration of the compound (a1) is preferably 1% by mass to 2% by mass.

On the other hand, in Comparative Examples 1 to 11 in which one or both of the additive corresponding to the compound (a1) and the additive corresponding to the compound (a2) were not used, there was no case in which all of the discharge capacity maintenance rate, the volume change (generation of gases), and the resistance increase rate were favorable.

That is, in Comparative Examples 1 and 2 in which 14BS or SL was used in the compound (a2), the discharge capacity maintenance rate was 70% or less, and the volume change was great (specifically, 2% or more).

In addition, Comparative Example 3 in which only MMDS was used as an additive, the volume change and the resistance increase rate were suppressed but the discharge capacity maintenance rate was decreased.

In addition, in Comparative Example 4 in which only PS was used as an additive, the capacity maintenance rate and the volume change were favorable, but the resistance increase rate was high. In Comparative Examples 5 to 8, the volume change and the resistance increase rate were great.

In addition, in Comparative Examples 9 to 11 in which another additive was used, the volume change was particularly big (specifically, there was more than 5% of volume change).

TABLE 3

| | | | | Capacity | | Resistance |
|---|---|---|---|---|---|---|
| | | | Other | maintenance | Volume | increase |
| | Additive 1 | Additive 2 | additive | rate | change | rate |
| Example 1 | MMDS 0.5% by | PS 1.5% by | — | GOOD | GOOD | GOOD |
| | mass | mass | | | | |
| Example 2 | MMDS 1.0% by | PS 1.5% by | — | GOOD | GOOD | GOOD |
| | mass | mass | | | | |
| Example 3 | MMDS 1.5% by | PS 1.5% by | — | GOOD | GOOD | GOOD |
| | mass | mass | | | | |
| Example 4 | MMDS 2.0% by | PS 1.5% by | — | GOOD | GOOD | GOOD |
| | mass | mass | | | | |
| Example 5 | MMDS 1.5% by | 24BS 1.5% by | — | GOOD | GOOD | GOOD |
| | mass | mass | | | | |
| Example 6 | MMDS 1.5% by | TMS 1.5% by | — | GOOD | GOOD | GOOD |
| | mass | mass | | | | |
| Comparative | MMDS 1.5% by | 14BS 1.5% by | — | POOR | POOR | GOOD |
| Example 1 | mass | mass | | | | |
| Comparative | MMDS 1.5% by | SL 1.5% by | — | POOR | POOR | GOOD |
| Example 2 | mass | mass | | | | |
| Comparative | MMDS 3.0% by | — | — | POOR | GOOD | GOOD |
| Example 3 | mass | | | | | |
| Comparative | — | PS 3.0% by | — | GOOD | GOOD | POOR |
| Example 4 | | mass | | | | |
| Comparative | — | SL 3.0% by | — | GOOD | POOR | POOR |
| Example 5 | | mass | | | | |
| Comparative | — | 14BS 3.0% by | — | POOR | POOR | POOR |
| Example 6 | | mass | | | | |
| Comparative | — | 24BS 3.0% by | — | GOOD | POOR | POOR |
| Example 7 | | mass | | | | |
| Comparative | — | TMS 3.0% by | — | GOOD | POOR | POOR |
| Example 8 | | mass | | | | |
| Comparative | — | — | VC 3.0% by | GOOD | POOR | GOOD |
| Example 9 | | | mass | | | |
| Comparative | — | — | FEC 3.0% by | GOOD | POOR | POOR |
| Example 10 | | | mass | | | |
| Comparative | — | — | SA 3.0% by | GOOD | POOR | POOR |
| Example 11 | | | mass | | | |

Priority is claimed on Japanese Patent Application No. 2017-087254, filed on Apr. 26, 2017, the content of which is incorporated herein by reference.

1. A lithium ion secondary battery comprising:
a positive electrode including a positive electrode active material containing a lithium nickel complex oxide;
a negative electrode; and
an electrolyte,
wherein the electrolyte includes a cyclic sulfonic acid ester (a1) which contains at least two sulfonyl groups in a molecule and a compound (a2) which contains only one sulfonyl group in a molecule and of which an energy level of a highest occupied molecular orbital calculated by a PM3 method is −11.2 eV or less.

2. A lithium ion secondary battery comprising:
a positive electrode including a positive electrode active material containing a lithium nickel complex oxide;
a negative electrode; and
an electrolyte,
wherein a film containing a sulfur atom is present on at least a portion of a surface of the positive electrode active material, and
wherein the electrolyte includes a compound (a2) which contains only one sulfonyl group in a molecule and of which an energy level of a highest occupied molecular orbital calculated by a PM3 method is −11.2 eV or less.

3. The lithium ion secondary battery according to claim 1,
wherein a concentration of the compound (a2) is 1.0% by mass to 6.0% by mass based on a total amount of the electrolyte.

4. The lithium ion secondary battery according to claim 1,
wherein the compound (a2) is a compound of which an energy level of a lowest unoccupied molecular orbital calculated by a PM3 method is 0 to 0.2 eV.

5. The lithium ion secondary battery according to claim 1,
wherein the compound (a2) has a ring structure, and contains only one —SO₂-structure in the ring structure.

6. The lithium ion secondary battery according to claim 1,
wherein the lithium nickel complex oxide includes at least one element selected from the group consisting of cobalt and aluminum.

7. The lithium ion secondary battery according to claim 1,
wherein the lithium nickel complex oxide is represented by a composition formula LiₓNi_{1-y}M_{y}O₂ (M includes at least one metal selected from the group consisting of Co, Fe, Ti, Cr, Mg, Al, Cu, Ga, Mn, Zn, Sn, B, V, Ca, and Sr, and satisfies 0.05≤x≤1.2, 0≤y≤0.5).

8. A manufacturing method of the lithium ion secondary battery according to claim 2, the method comprising:
a step of assembling a non-charged lithium ion secondary battery including (i) an electrolyte including a compound (a1) which contains two or more sulfonyl groups in a molecule and a compound (a2) which contains only one sulfonyl group in a molecule and of which an energy level of a highest occupied molecular orbital calculated by a PM3 method is −11.2 eV or less, (ii) a positive electrode including a positive electrode active material containing a lithium nickel complex oxide, and (iii) a negative electrode; and
a step of performing charge on the non-charged lithium ion secondary battery, reacting the positive electrode active material with the compound (a1) included in the electrolyte, and forming a film containing a sulfur atom on at least a portion of the surface of the positive electrode active material.

9. An electrolyte for a lithium ion secondary battery comprising:
a positive electrode including a positive electrode active material containing a lithium nickel complex oxide;
a compound (a1) which contains two or more sulfonyl groups in a molecule; and
a compound (a2) which contains only one sulfonyl group in a molecule and of which an energy level of a highest occupied molecular orbital calculated by a PM3 method is −11.2 eV or less.

10. The lithium ion secondary battery according to claim 2,
wherein a concentration of the compound (a2) is 1.0% by mass to 6.0% by mass based on a total amount of the electrolyte.

11. The lithium ion secondary battery according to claim 2,
wherein the compound (a2) is a compound of which an energy level of a lowest unoccupied molecular orbital calculated by a PM3 method is 0 to 0.2 eV.

12. The lithium ion secondary battery according to claim 2,
wherein the compound (a2) has a ring structure, and contains only one —SO₂-structure in the ring structure.

13. The lithium ion secondary battery according to claim 2,
wherein the lithium nickel complex oxide includes at least one element selected from the group consisting of cobalt and aluminum.

14. The lithium ion secondary battery according to claim 2,
wherein the lithium nickel complex oxide is represented by a composition formula LiₓNi_{1-y}M_{y}O₂ (M includes at least one metal selected from the group consisting of Co, Fe, Ti, Cr, Mg, Al, Cu, Ga, Mn, Zn, Sn, B, V, Ca, and Sr, and satisfies 0.05≤x≤1.2, 0≤y≤0.5).
